(19) United States
(12) Patent Application Publication
(10) Pub. No.: US 2022/0384024 A1
(43) Pub. Date: Dec. 1, 2022

(54) SYSTEM FOR ADMINISTERING A MEDICAL FLUID TO A PATIENT

(71) Applicant: Fresenius Vial SAS, Brézins (FR)

(72) Inventors: Olivier Artarit, St Etienne De Crossey (FR); Pascal Bricard, Grenoble (FR)

(21) Appl. No.: 17/758,516

(22) PCT Filed: Feb. 19, 2021
(86) PCT No.: PCT/EP2021/054093

§ 371 Date: Jul. 8, 2022

(30) Foreign Application Priority Data
Feb. 20, 2020 (EP) 20290021.3

(51) Int. Cl. G16H 40/40; G16H 40/63

(52) U.S. Cl. CPC G16H 40/40; G16H 40/63; G16H 20/17

(57) ABSTRACT

A system for administering a medical fluid to a patient (P), comprises an infusion station (1) comprising a rack (10) defining a multiplicity of slots (100) for receiving a multiplicity of infusion devices (2), a first processing device (13, 40) associated with the infusion station (1) and configured to execute a first software application (130, 400) being associated with a first version identifier, and a second processing device (23) associated with the infusion station (1) and configured to execute a second software application (230) being associated with a second version identifier. At least one of the first processing device (13, 40) and the second processing device (23) is configured to store a data structure (131, 231 401) comprising compatibility information, wherein at least one of the first processing device (13, 40) and the second processing device (23) is configured to perform a compatibility check of the first software application (130, 400) and the second software application (230) with each other based on the first version identifier, the second version identifier and the data structure (131, 231), the compatibility information comprising a list of software versions which are compatible or not compatible with the first software application and/or the second software application.

### DESCRIPTION

The invention relates to a system for administering a medical fluid to a patient according to the preamble of claim 1 and to a method for administering a medical fluid to a patient.

A system of this kind comprises an infusion station comprising a rack defining a multiplicity of slots for receiving a multiplicity of infusion devices. The system furthermore comprises a first processing device associated with the infusion station and configured to execute a first software application being associated with a first version identifier, and a second processing device associated with the infusion station and configured to execute a second software application being associated with a second version identifier.

A system of this kind may generally be used at the bedside of a patient in a healthcare environment, for example within an intensive care unit (ICU) of a hospital. Using an infusion station of the type described herein a multiplicity of infusion devices, such as syringe infusion pumps or volumetric (peristaltic) infusion pumps, may be combined in order to administer one or multiple medical fluids, such as medications for the treatment of the patient or nutritional solutions for the enteral or parenteral feeding of the patient, to a patient in a concerted fashion, for example within an infusion protocol in which infusion operations are carried out sequentially or in parallel. Furthermore, using the infusion station infusion devices may be organized and physically held to form for example a stack of infusion devices at the bedside of the patient, such that an organized and easy-to-handle arrangement of infusion devices is obtained. The rack herein serves as a communication link in that it links infusion devices to each other and to the control device, the rack hence serving as a communication spine which in addition may provide for a power supply for electrically feeding the infusion devices connected thereto.

Generally, a particular infusion station is used to perform infusion operations on a single patient. Multiple infusion devices arranged on the rack herein shall interwork for performing infusion operations on the patient, making it necessary that the infusion devices, in their operation, are compatible with one another such that a correct functioning of the infusion devices is ensured.

Infusion devices as well as a control device of the rack may comprise processing devices configured to execute software applications. In particular, infusion devices may run software applications for performing infusion operations, such software applications providing for an operating system to control different functions of a particular infusion device, for example for controlling a pumping mechanism or other devices such as sensing devices or the like, for evaluating data arising during an infusion operation, for communicating with other devices, for outputting information and/or for allowing a programming of infusion operations. Likewise, a control device of the rack may run a software application, the control device providing for a link for example to a hospital information system and for interconnecting infusion devices connected to the rack to each other.

In an infusion station comprising a rack having multiple slots for receiving multiple infusion devices it is desirous to perform a compatibility check between infusion devices attached to the rack and the rack itself in order to make sure that an infusion station equipped with a multiplicity of infusion devices may function correctly. It herein is to be expected that multiple infusion devices may run different versions of software applications, such that not all infusion devices necessarily use the same software application, in particular the same version of a software application. Nevertheless, a correct interworking must be ensured.

US2008097168 relates to a medical workstation with a therapy module in which the control and display unit has an operating program, which is designed such that the area on the control and display unit is able to be operated by the user only in case of connection of a compatible therapy module. The control and display unit checks the compatibility of its program version with that of the therapy module.

It is an object of the instant invention to provide a system for administering a medical fluid to a patient and a method for operating such a system which allow for an easy and reliable compatibility check for enabling an interworking of devices of the infusion station.

This object is achieved by means of a system comprising the features of claim 1.

Accordingly, the at least one of the first processing device and the second processing device is configured to store a data structure comprising compatibility information, wherein at least one of the first processing device and the second processing device is configured to perform a compatibility check of the first software application and second software application with each other based on the first version identifier, the second version identifier and the data structure, preferably the compatibility information comprising a list of software versions which are compatible or not compatible with said first software application and/or said second software application.

In one embodiment, the data structure hence may have a list structure in which a compatibility or non-compatibility in between software versions of the first software application and the second software application is identified. By checking the first version identifier and/or the second version identifier versus the data structure, hence, a compatibility check may be carried out for checking a compatibility of the first software application and the second software application.

Based on the compatibility check the system is enabled to ensure a compatibility in between devices of the infusion station. The compatibility check herein takes place in that a version identifier of a first software application to be executed by a first processing device and a second version identifier of a second software application to be executed by a second processing device are checked and verified versus a data structure comprising compatibility information associated with the first processing device and/or the second processing device. By for example checking the first version identifier and the second version identifier versus the data structure it can be made sure that the first software application identified by the first version identifier in fact is compatible with the second software application identified by the second version identifier.

In one embodiment, the infusion station comprises a control device comprising said first processing device. The control device may for example be connected to the rack and may provide for a link of the infusion station to a hospital information system. In addition, the control device may be connected to a nurse call system and a reading device, such as a barcode reader. The control device also may be configured to control infusion operations carried out by infusion devices attached to the rack, the control device for example being configured for controlling a medical protocol for a multichannel infusion involving a multiplicity of infusion devices.

In this case, in one embodiment, at least one infusion device to be the attached to one of said multiplicity of slots of the rack may comprise said second processing device.

In another embodiment, a server, for example of an external hospital information system, may comprise the first processing device. In this case the first processing device is external to the infusion station, but is associated with the infusion station in that it executes a software application relating to the infusion station. The software application in this case for example may represent a maintenance software for performing maintenance operations on the infusion station, for example on infusion devices connected to the rack of the infusion station, for example for performing a software updates, calibration operations, a maintenance configuration, a functional check, a backup, or a factory reset.

In this case, the second processing device is for example part of a control device of the infusion station, such that the compatibility check is carried in between the processing device of the server and the processing device of the control device.

In another embodiment, at least one infusion device to be the attached to one of said multiplicity of slots of the rack may comprise said second processing device, such that the compatibility check is carried in between the processing device of the server and the processing device of the infusion device.

If the first processing device for example is part of the control device of the rack, and the second processing device is part of an infusion device to be attached to the rack, the compatibility check hence is performed between the software application executed on the control device and the software application executed on the infusion device.

If the first processing device instead is part of an external server, and the second processing device is part of an infusion device to be attached to the rack of the infusion station, the compatibility check is performed between the software application executed on the external server and the software application executed on the infusion device, the software application to be executed on the external server for example being a maintenance software for performing a maintenance operation on the infusion device.

In another embodiment, the first processing device may also be part of an infusion device, wherein the second processing device is part of another infusion device. In this case, the compatibility check is performed between two infusion devices attached to the rack of the infusion station.

If the second processing device is part of an infusion device to be attached to the rack of the infusion station, the compatibility check may for example be performed once the infusion device is attached to the rack. In this case, in one embodiment, the second processing device is configured to transfer the second version identifier and potentially also a data structure to the first processing device on the occasion of attaching the infusion device to the rack. The first processing device, which for example as part of the control device of the rack, is hence enabled to perform the compatibility check, such that it may be verified that the version of the software application executed on the infusion device in fact is compatible with the software application as executed on the control device.

In particular, at least one of the first processing device and the second processing device may be configured to examine, for performing the compatibility check, whether the first version identifier and/or the second version identifier is identified in the data structure. The data structure herein may for example be of the type of a blacklist in which non-compatibilities are identified, such that it may be checked according to the first version identifier and the second version identifier whether a non-compatibility for a particular software version exists.

In one embodiment, the first processing device is configured to store a first data structure comprising first compatibility information and the second processing device is configured to store a second data structure comprising second compatibility information, wherein at least one of the first processing device and the second processing device is configured to perform a compatibility check of the first software application and the second software application with each other based on the first version identifier, the second version identifier, the first data structure and the second data structure.

The compatibility check hence, according to this embodiment, takes place in that a version identifier of a first software application to be executed by a first processing device and a second version identifier of a second software application to be executed by a second processing device are checked and verified versus a first data structure comprising first compatibility information associated with the first processing device and a second data structure comprising second compatibility information associated with the second processing device. By checking the first version identifier for example versus the second data structure and by checking in addition the second version identifier versus the first data structure it can be made sure that the first software application identified by the first version identifier in fact is compatible with the second software application identified by the second version identifier.

In one embodiment, the first compatibility information of the first data structure comprises a first amount of identifier information. The first amount of identifier information may for example be a list of software versions which are compatible or are not compatible with the first software application.

Likewise, the second compatibility information of the second data structure may comprise a second amount of identifier information. The second of amount of identifier information may for example be a second list of software versions which are compatible or are not compatible with the second software application.

Generally, in a system of the kind described herein, it is assumed that all devices of a particular manufacturer shall be compatible with one another. Hence, it generally shall be assumed that software applications executed on devices of a system comprising an infusion station of the type as described herein shall be compatible to interwork with each other and to communicate with each other to ensure a proper functioning of infusion devices attached to a rack of an infusion station.

There however may be exceptions in that particular software versions of a software application executed on one processing device may not be compatible with a particular software version of a software application executed on another processing device. Hence, in the first data structure and likewise in the second data structure identifier information may be stored which indicate whether a particular version of a software application executed on one processing device is compatible or not with a particular version of a software application executed on another processing device.

As it may be the case that a version of a software application, as executed for example on a processing device of the control device of the rack, from its own perspective is enabled to interwork with a version of a software application executed on an infusion device and hence is compatible with the software application of the infusion device, but in turn the version of the software application executed on the infusion device from its perspective is not compatible with the version of the software application executed on the control device, in one embodiment two data structures comprising different compatibility information are present and used for the compatibility check.

In particular, the first data structure by means of its compatibility information, for example in the shape of a list of software versions, identifies such software versions which, in one embodiment, are not compatible with the version of the first software application (or, in an alternative embodiment, which are compatible with the version of the first software application). The list of software versions for example may represent a blacklist which indicates all those versions with which the software application of the first processing device cannot interwork (or alternatively a whitelist indicating all those versions with which the software application of the second processing device can interwork).

Likewise, the second data structure by means of its compatibility information, for example in the shape of a list of software versions, identifies such software versions which, in one embodiment, are not compatible with the version of the second software application (or, in an alternative embodiment, which are compatible with the version of the second software application). The list of software versions for example may represent a blacklist which indicates all those versions with which the software application of the second processing device cannot interwork (or alternatively a whitelist indicating all those versions with which the software application of the second processing device can interwork).

In one embodiment, at least one of the first processing device and the second processing device is configured to examine, for performing the compatibility check, whether the first version identifier is identified in the second data structure and/or the second version identifier is identified in the first data structure. For example, it may be checked whether the first version identifier is part of a list of software versions as stored in the second data structure, and in addition whether the second version identifier is part of a list of software versions stored in the first data structure. Based on this twofold check it then is determined whether the software applications of the processing devices are compatible with each other.

For example, an interworking of the first software application and the second software application may be enabled if and only if the first version identifier is not identified in the second data structure and the second version identifier is not identified in the first data structure as being not compatible. Hence, in this case, the first data structure and the second data structure represent a blacklist identifying those software versions with which the respective other software application is not compatible. Only if the version identifier of the software applications are not identified in the respective other data structure, a compatibility in between the software applications is assumed and the software applications hence are enabled to interwork with each other.

The enablement for interworking may in particular involve a communication in between the software applications. In the course of the communication, data may be exchanged in between the software applications, such that for example a synchronization in between an infusion device attached to the rack and a control device of the rack may be achieved.

The compatibility check may be performed on either of the processing devices, or on both processing devices together. For example, the first processing device may transfer its version identifier to the second processing device, such that the second processing device may check for a compatibility according to its data structure. Likewise, the second processing device may send its version identifier to the first processing device, such that the first processing device is enabled to check for compatibility based on its data structure.

The object is also achieved by a method for operating a system for administering a medical fluid to a patient, the method comprising: providing an infusion station comprising a rack defining a multiplicity of slots for receiving a multiplicity of infusion devices; providing a first processing device associated with the infusion station and configured to execute a first software application being associated with a first version identifier; and providing a second processing device associated with the infusion station and configured to execute a second software application being associated with a second version identifier. Herein, the method further comprises: performing a compatibility check of the first software application and the second software application with each other based on the first version identifier, the second version identifier and a data structure, the data structure comprising compatibility information and being stored by at least one of the first processing device and the second processing device.

The advantages and advantageous embodiments described above for the system equally apply also to the method, such that it shall be referred to the above.

The idea underlying the invention shall subsequently be described in more detail with reference to the embodiments shown in the figures. Herein:

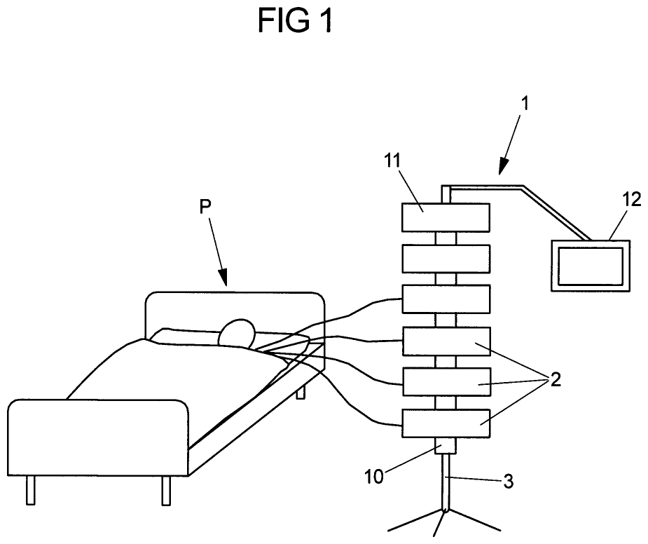

FIG. 1 shows a schematic drawing of an infusion station at the bedside of a patient;

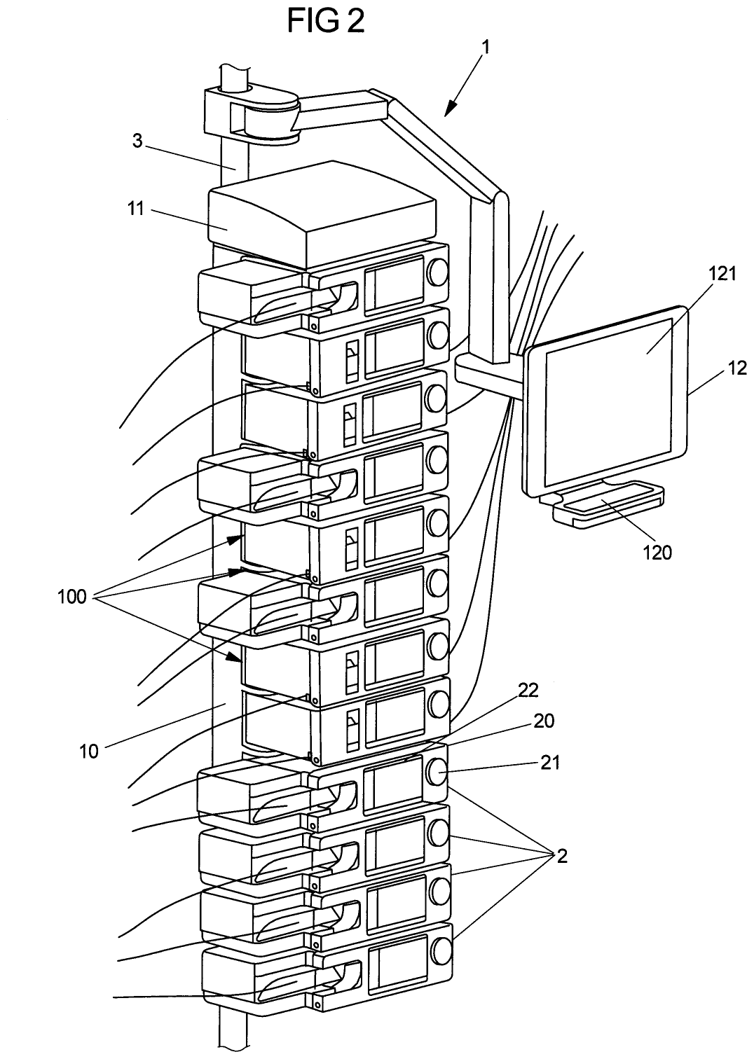

FIG. 2 shows a view of an embodiment of an infusion station;

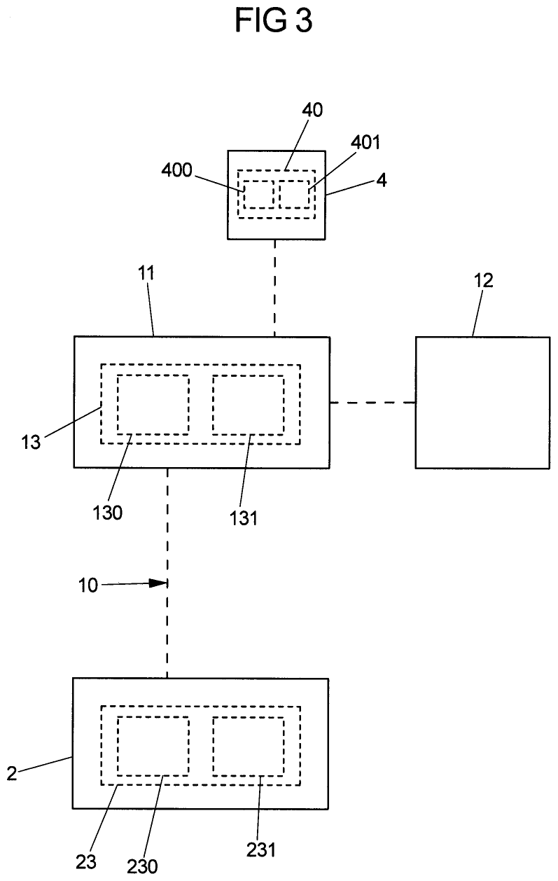

FIG. 3 shows a schematic diagram of the interaction of a control device and an infusion device of an infusion station;

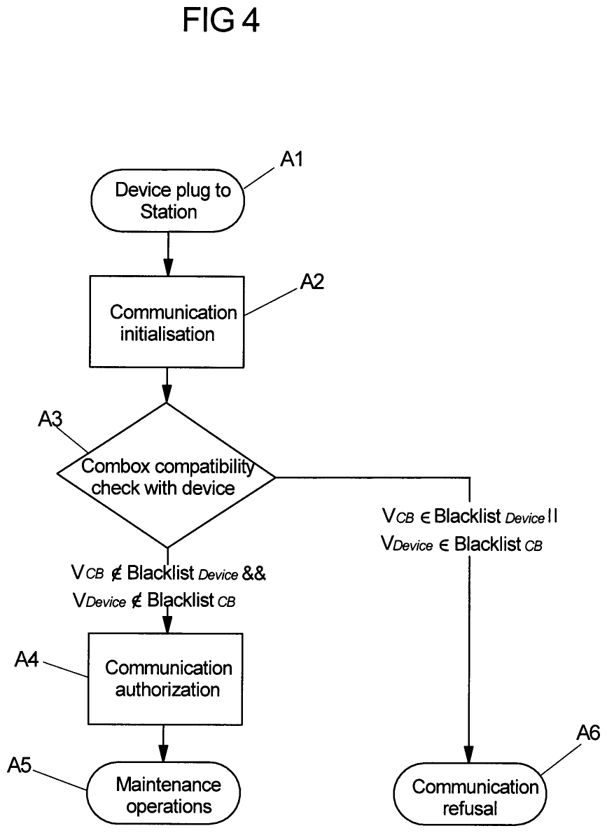

FIG. 4 a flowchart for performing a compatibility check when attaching an infusion device to a rack of an infusion station, according to one embodiment; and

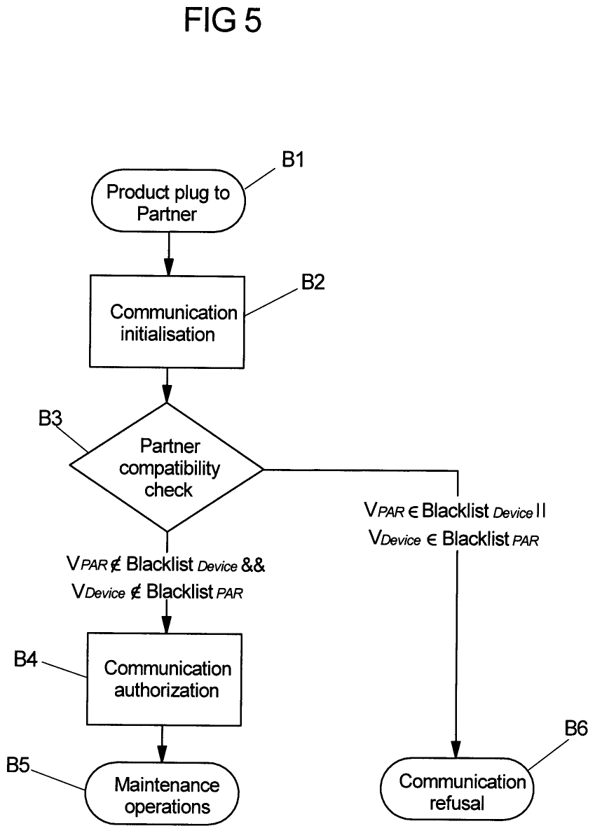

FIG. 5 a flowchart for performing a compatibility check in between a maintenance application (denoted as Partner) and an infusion device, according to one embodiment.

FIG. 1 shows a schematic drawing of an infusion station 1 at the bedside of a patient P, for example in a healthcare environment, such as in an intensive care unit (ICU) of a hospital.

As visible from FIG. 1 in view of FIG. 2, the infusion station 1 comprises a rack 10 defining a multiplicity of slots 100 to which a multiplicity of infusion devices 2 may be attached. The rack 10, at the slots 100, provides for a mechanical and electrical connection for the infusion devices 2, the rack 10 serving as a communication link as well as a power supply such that the infusion devices 2 connected to the rack 10 are interconnected with each other and further are connected to a power supply via the rack 10.

The rack 10 comprises a control device 11 which, as schematically shown in FIG. 3, comprises a processing device 13 and hence comprises electronic circuitry for controlling operation of the infusion station 1. The control device 11 in particular provides for a connection to a server 4 of a hospital information system, such that via the control device 11 data may be transferred to and may be received from the hospital information system.

In particular, the control device 11 may be configured to send infusion data resulting from infusion operations carried out by infusion devices 2 of the infusion station 1 to the hospital information system. Alternatively or in addition, the control device 11 may be configured to receive configuration data, such as drug library data, from the hospital information system.

In addition, a nurse call system and a reading device such as a barcode reader or the like may be connected to the control device 11.

Alternatively or in addition, the control device 11 may be configured to control operation of the infusion devices 2 connected to the rack 10, for example for carrying out infusion operations in a concerted fashion, such as within a medical protocol for a multichannel infusion.

The rack 10 together with the control device 11 and the infusion devices 2 arranged thereon is connected to a stand 3 placed at the bedside of a patient P, as visible from FIG. 1. The rack 10 holds the infusion devices 2 connected thereto in an organized fashion, forming a vertical stack of infusion devices 2.

The infusion station 1 in addition comprises an interface device 12, also denoted as therapy manager, which is functionally connected to the control device 11—as indicated in FIG. 3—and comprises an input device 120 and a display 121. The interface device 12 is configured to output information about infusion operations carried out by infusion devices 2 of the infusion station 1. In addition, a user may enter information into the input device 120 for example for programming infusion operations of the infusion station 1.

As visible from FIG. 2, the infusion devices 2 may be configured as syringe infusion pumps or as volumetric (peristaltic) infusion pumps. In either case, the infusion devices 2 may comprise a housing 20, an input device 21 for example in the shape of a rotary knob and/or a keypad or the like for inputting data into the respective infusion device 2, for example for programming an infusion operation, and a display 22 for outputting information, for example relating to an ongoing infusion operation.

Referring again to FIG. 3, each infusion device 2 comprises a processing device 23 for controlling operation of the infusion device 2.

The infusion devices 2 arranged on the rack 10 of the infusion station 1 may be configured to individually or together in a multichannel operation perform infusions on a patient P to which the infusion station 1 is assigned. Herein, each infusion station 1 generally is assigned to a single patient P, the infusion devices 2 hence being dedicated to the patient P for carrying out infusion operations on the patient P.

Each infusion device 2 generally comprises a processing device 23 being configured to execute a software application 230 for operating the infusion device 2, in particular for performing infusion operations. The software application 230 in particular provides for an operating system allowing to control electrical and mechanical assemblies of the infusion device 2, such as a pumping mechanism or sensor devices, and to evaluate data, for example arising during an infusion operation, to allow for an effective control of the infusion device 2.

In addition, the control device 11 comprises a processing device 13 configured to execute a software application 130, the software application 130 for example being configured to allow for a communication with an external server 4 of a hospital information system, and for providing for a communication with infusion devices 2 attached to the rack 10 of the infusion station 1. In addition, the software application 130 may be programmed to conduct control operations, such as for performing a multichannel infusion operation involving multiple infusion devices 2.

Further in addition, the external server 4 may comprise a processing device 40 executing a software application 400, which may for example provide for a maintenance of the control device 11 and infusion devices 2 attached to the rack 10 of the infusion station 1, for example for the purpose of performing software updates, calibration operations (relating to a force sensor, rotary potentiometers, luminosity, daytime or the like), maintenance configurations (relating to product codes, versions, language, syringes database, technical parameters or the like), functional checks (occlusion detection tests or the like), along with pump parameters customizations (display settings, menu settings, drug library selection, infusion settings, syringes settings), a backup, and/or a factory reset.

Generally, the software applications 230 of the infusion devices 2, the software application 130 of the control device 11 and the software application 400 of the external server 4 shall interwork with each other in order to allow a correct functioning of the infusion station 1 including all infusion devices 2 attached thereto. Herein, it generally can be assumed that devices of a particular manufacturer are and able to interwork with each other and hence can be assumed to be compatible, wherein however exceptions with respect to particular software versions of a software application 130, 230, 400 may exist.

Hence, in particular on the occasion of attaching an infusion device 2 to one of the slots 100 of the rack 10 a compatibility check shall be performed in order to check for a compatibility of the software applications 130, 230, 400 with each other. Herein, it may be the case that different infusion devices 2 may operate on different software versions of a software application, which nevertheless in general shall not hinder operation of the infusion station 1, unless particular software versions for specific reasons are incompatible with each other.

Referring now to FIG. 4, a compatibility check may in particular be carried out on the occasion of attaching an infusion device 2 to one of the slots 100 of the rack 10 of the infusion station 1 to check for a compatibility of the software version of the software application 230 of the infusion device 2 and the version of the software application 130 of the control device 11.

As visible from FIG. 4, once an infusion device 2 is inserted into a slot 100 of the rack 10 (step A1), a communication in between the infusion device 2 and the control device 11 is initialized, in particular involving the software applications 130, 230 (step A2). Within this communication, in a data message, for example in the shape of a so-called topology message, the software application 230 may transfer information relating to a version identifier identifying the software version of the software application 230 and in addition a data structure 231 comprising compatibility information to the control device 11, namely to the software application 130 of the processing device 13 of the control device 11.

The software application 130 of the control device 11 likewise is identified by a version identifier, wherein the processing device 13 in addition stores a data structure 131 comprising compatibility information.

In the data structure 231 of the software application 230 of the infusion device 2 information is stored indicating which software versions are compatible or not with the software application 230 executed on the infusion device 2. In turn, in the data structure 131 of the software application 130 of the control device 11 information is stored indicating which software versions are compatible or not with the software application 130 executed on the control device 13.

The data structure 131 and the data structure 231 may for example have the shape, in each case, of a blacklist identifying those software versions which are not compatible with the respective software application 130, 230. Hence, in each data structure 131, 231 a list of version identifiers is stored indicating a potential incompatibility with a particular software version.

Within the compatibility check (step A3 in FIG. 4) it then is checked, in a twofold approach, whether the version identifier relating to the software application 130 of the control device 11 is identified in the data structure 231 relating to the software application 230 of the infusion device 2, and in addition whether the version identifier of the software application 230 is identified in the data structure 131 relating to the software application 130 of the control device 11. If each data structure 131, 231 has the shape of a blacklist, a compatibility in between the software applications 130, 230 is assumed if and only if none of the version identifiers is identified in the respective other data structure 131, 231. Hence, it is checked whether the version identifier of the software application 230 is an element of the blacklist relating to the software application 130, and whether the version identifier of the software application 130 is an element of the blacklist relating to the software application 230. Only if this is not the case, an interworking between the software applications 130, 230 is authorized (step A4) and a communication in between the software applications 130, 230 takes place (step A5).

If during the compatibility check in step A3 it is found that the software identifier of the software application 230 is identified in the data structure 131 relating to the software application 130, and/or the version identifier of the software application 130 is identified in the data structure 231 relating to the software application 230, it is found for an incompatibility between the software applications 130, 230, and hence an interworking is refused (step A6). The infusion device 2 hence is found to be incompatible with the infusion station 1 and hence is rejected, which can be output to a user for example by a warning message or the like on the display 22 of the infusion device 2.

Referring now to FIG. 5, upon the attachment of an infusion device 2 to a slot 100 of the rack 10, also a compatibility check in between the software application 400 of an external server 4 and the software application 230 of the infusion device 2, for example for the purpose of performing a maintenance operation, may be carried out. The procedure herein takes place in the same manner as described above with reference to FIG. 4.

In particular, on the occasion of attaching the infusion device 2 to the rack 10 (step B1), a communication in between the software application 230 of the infusion device 2 and the software application 400 executed on the server 4 is initialized (step B2). Then, a compatibility check is carried out (step B3), in which it is checked whether the version identifier of the software application 230 is identified in a data structure 401 relating to the software application 400 of the external server 4, and in addition whether the version identifier of the software application 400 is identified in the data structure 231 relating to the software application 230 of the infusion device 2.

Again, as described before according to FIG. 4, the data structure 231 and the data structure 401 may for example have the shape, in each case, of a blacklist identifying those software versions which are not compatible with the respective software application 230, 400. Hence, in each data structure 231, 401 a list of version identifiers is stored indicating a potential incompatibility with a particular software version. During the check it is examined whether the software application 230 is identified as non-compatible in the data structure 401 and the software application 400 is identified as non-compatible in the data structure 231. Only if this is not the case, an interworking in between the software applications 230, 400 is authorized (step B4) and for example a maintenance on the infusion device 2 may be carried out (step B5). If however one software version is identified in the data structure 231, 401 of the respective other device, it is assumed that the software applications 230, 400 are incompatible with one another, and an interworking in between the software applications 230, 400 is refused (step B6).

The idea underlying the invention is not limited to the embodiments described above, but may be implemented in a different fashion.

A compatibility check may be performed generally between any two devices of a system for administering a medical fluid to a patient, for example in between an infusion device and a control device of a rack, in between two infusion devices, or in between an infusion device and an external device, such as an external server.

The compatibility check may be carried out according to a blacklist, but likewise may also be carried out according to a whitelist identifying software versions which in fact are compatible.

### LIST OF REFERENCE NUMERALS

1 Infusion station
10 Rack
100 Slot
11 Control device
12 Interface device
120 Input device
121 Display
13 Processing device
130 Software application
131 Data structure (compatibility register)
2 Infusion device
20 Housing
21 Input device
22 Display
23 Processing device
230 Software application
231 Data structure (compatibility register)
3 Stand
4 Server (hospital information system)
40 Processing device
400 Software application (maintenance application)
401 Data structure (compatibility register)
A1-A6 Steps
B1-B6 Steps
P Patient

1. A system for administering a medical fluid to a patient, comprising:
an infusion station comprising a rack defining a multiplicity of slots for receiving a multiplicity of infusion devices,
a first processing device associated with the infusion station and configured to execute a first software application being associated with a first version identifier, and
a second processing device associated with the infusion station and configured to execute a second software application being associated with a second version identifier,
wherein at least one of the first processing device and the second processing device is configured to store a data structure comprising compatibility information, and at least one of the first processing device and the second processing device is configured to perform a compatibility check of the first software application and the second software application with each other based on the first version identifier, the second version identifier and the data structure and in that the compatibility information comprises a list of software versions which are compatible or not compatible with the first software application and/or the second software application.

2. The system according to claim 1, wherein the infusion station comprises a control device comprising said first processing device.

3. The system according to claim 2, further comprising at least one infusion device to be attached to one of said multiplicity of slots of the rack, the at least one infusion device comprising said second processing device.

4. The system according to claim 1, further comprising a server external to the infusion station and comprising said first processing device.

5. The system according to claim 4, wherein the infusion station comprises a control device comprising said second processing device.

6. The system according to claim 4, further comprising at least one infusion device to be attached to one of said multiplicity of slots of the rack, the at least one infusion device comprising said second processing device.

7. The system according to claim 6, wherein the second processing device is configured, on an occasion of attaching the at least one infusion device to one of the multiplicity of slots of the rack, to transfer said second version identifier to the first processing device to enable the first processing device to perform said compatibility check.

8. The system according to claim 1, wherein said at least one of the first processing device and the second processing device is configured to examine, for performing the compatibility check, whether the first version identifier and/or the second version identifier is identified in the data structure.

9. The system according to claim 1, wherein the first processing device is configured to store a first data structure comprising first compatibility information and the second processing device is configured to store a second data structure comprising second compatibility information, and at least one of the first processing device and the second processing device is configured to perform a compatibility check of the first software application and the second software application with each other based on the first version identifier, the second version identifier, the first data structure and the second data structure.

10. The system according to claim 9, wherein the first compatibility information comprises a first amount of identifier information, wherein the first amount of identifier information is a first list of software versions which are compatible or not compatible with the first software application.

11. The system according to claim 9, wherein the second compatibility information comprises a second amount of identifier information, wherein the second amount of identifier information is a second list of software versions which are compatible or not compatible with the second software application.

12. The system according to claim 9, wherein said at least one of the first processing device and the second processing device is configured to examine, for performing the compatibility check, whether the first version identifier is identified in the second data structure and/or the second version identifier is identified in the first data structure.

13. The system according to claim 12, wherein said at least one of the first processing device and the second processing device is configured to enable an interworking of the first software application and the second software application if and only if the first version identifier is not identified in the second data structure and the second version identifier is not identified in the first data structure as being not compatible.

14. A method for operating a system for administering a medical fluid to a patient, comprising:
providing an infusion station comprising a rack defining a multiplicity of slots for receiving a multiplicity of infusion devices,
providing a first processing device associated with the infusion station and configured to execute a first software application being associated with a first version identifier, and
providing a second processing device associated with the infusion station and configured to execute a second software application being associated with a second version identifier, and
performing a compatibility check of the first software application and the second software application with each other based on the first version identifier, the second version identifier and a data structure, the data structure comprising compatibility information and being stored by at least one of the first processing device and the second processing device.
